(19) United States
(12) Patent Application Publication
(10) Pub. No.: US 2024/0225561 A1
(43) Pub. Date: Jul. 11, 2024

(54) MONITORING OF UPPER LIMB MOVEMENTS TO DETECT STROKE

(71) Applicant: The Trustees of the University of Pennsylvania, Philadelphia, PA (US)

(72) Inventors: James Erich Weimer, Arlinton, VA (US); Steven Russell Messé, Wynnewood, PA (US)

(21) Appl. No.: 18/563,707

(22) PCT Filed: May 19, 2022
(86) PCT No.: PCT/US2022/030092

§ 371 Date: Nov. 22, 2023

Related U.S. Application Data
(60) Provisional application No. 63/193,053, filed on May 25, 2021.

(51) Int. Cl. A61B 5/00; A61B 5/11

(52) U.S. Cl. CPC A61B 5/7282; A61B 5/11; A61B 5/6824; A61B 5/7207; A61B 5/742; A61B 5/746; A61B 2562/0219

(57) ABSTRACT

Methods, systems, and computer readable media for detecting stroke by monitoring of upper limb movements. In some examples, a method for detecting stroke includes receiving, at a stroke detector implemented on at least one processor, movement data from an accelerometer attached to an upper limb of a patient for a period of time. The method includes analyzing, at the stroke detector, the movement data using a test statistic robust to motion distribution covariate shift to enable passive monitoring of the patient. The method includes outputting, at the stroke detector, an alarm signal in response to detecting a stroke using the movement data.

### PRIORITY CLAIM

This application claims the benefit of U.S. Patent Application Ser. No. 63/193,053, filed May 25, 2021, the disclosure of which is incorporated by reference in its entirety.

### GOVERNMENT INTEREST

This Invention was made with government support under 1915398 awarded by the National Science Foundation and FA8750-18-C-0090 awarded by the Air Force Research Laboratory. The government has certain rights in the invention.

### TECHNICAL FIELD

This specification relates generally to detecting stroke by monitoring of upper limb movements.

### BACKGROUND

Eligibility for stroke treatment and the likelihood of a good response to treatment are related to how quickly the stroke is identified. Stroke in already hospitalized patients is associated with delayed symptom detection and assessment, fewer interventions, and worse outcomes compared to strokes in the community.

Asymmetric arm strength and movement is one of the most common manifestations of acute stroke. Unfortunately, patients cannot be examined frequently enough to routinely detect stroke early after onset and allow for proven but time-limited interventions. Accordingly, there exists a need for automated methods, systems, and computer readable media configured for rapidly detecting stroke.

### SUMMARY

This specification describes methods, systems, and computer readable media for detecting stroke by monitoring of upper limb movements, e.g., by monitoring for asymmetric movement. In some examples, a method for detecting stroke includes receiving, at a stroke detector implemented on at least one processor, movement data from an accelerometer attached to an upper limb of a patient for a period of time. The method includes analyzing, at the stroke detector, the movement data using a test statistic robust to motion distribution covariate shift to enable passive monitoring of the patient without knowing any specific information about each patient (e.g., handedness). The method includes outputting, at the stroke detector, an alarm signal in response to detecting a stroke using the movement data.

In some examples, analyzing the movement data using the test statistic comprises analyzing the movement data using parameter invariant (PAIN) statistics. The test statistic can be, for example, a Komogorov-Smirnov statistic.

In some examples, receiving the movement data comprises receiving the movement data by a first wireless signal from a first wrist-mounted accelerometer on a first wrist of the patient. Receiving the movement data can include receiving a second wireless signal from a second wrist-mounted accelerometer on a second wrist of the patient. Receiving the movement data can include pre-processing the movement data to remove the effect of rotation/sliding of the accelerometer and bias.

In some examples, outputting the alarm signal comprises displaying an alarm message on a display screen. Outputting the alarm signal can include sending a message to a mobile device of a caregiver.

The subject matter described herein may be implemented in hardware, software, firmware, or any combination thereof. As such, the terms “function” or “node” as used herein refer to hardware, which may also include software and/or firmware components, for implementing the feature(s) being described. In some exemplary implementations, the subject matter described herein may be implemented using a computer readable medium having stored thereon computer executable instructions that when executed by the processor of a computer control the computer to perform steps.

Exemplary computer readable media suitable for implementing the subject matter described herein include non-transitory computer readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF DRAWINGS

FIG. 1A is a block diagram of an example system for stroke detection;

FIG. 1B is a block diagram illustrating an example configuration of the system for alerting a caregiver; and

FIGS. 2A-2D display the median and interquartile range (IQR) for the percentage of stroke cases that alarm as monitoring time increases using two different alarm thresholds;

FIG. 3 is a flow diagram illustrating an example method for detecting stroke.

### DETAILED DESCRIPTION

This specification describes methods, systems, and computer readable media for detecting stroke by monitoring of upper limb movements. The stroke detection systems described in this document allow for continuous monitoring of patients to detect stroke with lateralized weakness faster than usual care, which could lead to more and earlier stroke interventions and improved outcomes.

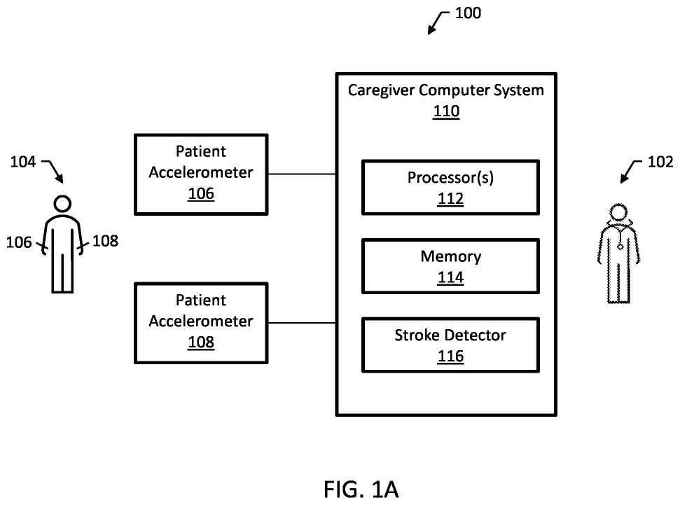

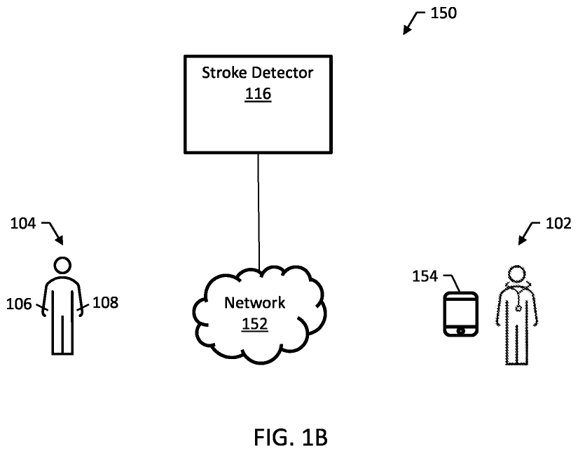

FIG. 1A is a block diagram of an example system 100 for stroke detection. The system 100 can be deployed, for example, in a hospital, or in any appropriate setting for detecting stroke. A caregiver 102 is providing medical attention to a patient 104. The caregiver 102, however, may not always be present with the patient 104.

Two accelerometers 106 and 108 are attached to upper limbs of the patient 104. For example, the first accelerometer 106 can be worn around a first wrist of the patient 104, and the second accelerometer 108 can be worn around a second wrist of the patient 104.

A caregiver computer system 110 includes at least one processor 112 and memory 114 storing executable instructions for the processor 112. Caregiver computer system 110 can be, for example, a tablet, laptop, or phone with a display device and speaker for alerting the caregiver 102. The caregiver computer system 110 includes a stroke detector 116 implemented using the processor 112 and memory 114.

The stroke detector 116 is configured for passively monitoring the patient 104 by receiving movement data from the accelerometers 106 and 108. The monitoring is passive in that the caregiver 102 need not ever give any instructions to the patient 104 to perform specific physical movements. For example, the stroke detector 116 can monitor for asymmetric arm movement.

The stroke detector 106 analyzes the movement data using a test statistic robust to motion distribution covariate shift to enable passive monitoring of the patient. The stroke detector 106 outputs an alarm signal in response to detecting a stroke using the movement data. For example, the stroke detector 106 can display an alarm image and/or play an alarm sound from the caregiver computer system 110, or the stroke detector 106 can transmit a signal to a remote computer system.

FIG. 1B is a block diagram illustrating an example configuration 150 of the system for alerting the caregiver 102. In this example, the accelerometers 106 and 108 are configured to transmit movement data to the stroke detector 116 by way of a data communications network 152, e.g., by transmitting to a wireless router to the Internet. The stroke detector 116 can, for example, be executing on a cloud computing server.

The stroke detector 116, in response to detecting stroke, outputs an alarm signal by transmitting to a caregiver device 154, e.g., a phone, tablet, or laptop. For example, the stroke detector 116 can send a text message to the caregiver device 154. The caregiver 102 can then provide appropriate medical care to the patient 104.

In general, the system can be configured in any appropriate way to alert the caregiver 102. For example, the accelerometers 106 and 108 could be included in wrist-mounted computer systems configured to execute the stroke detector 116, which can then output an alarm signal by playing an audio alert.

The methods and systems for stroke detection are described further below with respect to a study performed on the methodology.

### Introduction

Proven stroke treatments including intravenous thrombolysis and mechanical thrombectomy are highly time dependent. Eligibility for intervention and the probability of good outcome if treated decline continuously as time from onset of symptoms increases.¹⁻³ Thus, rapid detection of the onset of stroke symptoms is of paramount importance.⁴⁻⁶

Of the 800,000 strokes that occur annually in the United States, 5-17% develop in patients who are already hospitalized, the majority in patients who recently underwent an intervention or procedure.⁷⁻⁹ Compared to strokes that occur in the community, in-hospital stroke is associated with delayed detection and assessment, fewer interventions, and worse outcomes.⁷⁻¹¹ Thus, these complications lead to markedly increased cost, length of stay, morbidity, mortality, and medicolegal liability for hospitals and caregivers.^{7,8,10-14}

Upper extremity weakness is one of the most common findings in acute stroke.¹⁵ As a result, asymmetric arm strength is used in all screening tools for stroke.^{16,17} In addition, neglect is a frequent stroke symptom that also leads to a tendency to move the arm less on the affected side.^{18,19} This document describes an alerting system for automated monitoring for asymmetric arm movement that can be used in conjunction with wrist-worn accelerometers to rapidly identify stroke in hospitalized patients, facilitate more and earlier acute stroke treatments, and improve outcomes.

### Methods

We performed a prospective case-control study of upper extremity limb movements of patients admitted to the Hospital of the University of Pennsylvania in order to derive and validate a stroke detection algorithm. The study was approved by the Institutional Review Board at the Hospital of the University of Pennsylvania.

### Subjects

All subjects were recruited from the inpatient setting. Controls were neurologically normal with no history of stroke and included transient ischemic attack (TIA) patients without acute infarct on magnetic resonance imaging (MRI); patients undergoing work up of transient spells of uncertain etiology with normal MRI; and patients who recently underwent Cardiothoracic surgery or Vascular Surgery without overt neurologic complications. Cases included patients admitted with acute ischemic or hemorrhagic stroke with a National Institutes of Health Stroke Scale (NIHSS) score ≥1 and lateralizing limb weakness (at least 1 point for upper extremity weakness on item 5a or 5b of the NIHSS, greater on the affected side). Prior to the initiation of monitoring, subjects would undergo a neurologic evaluation including the NIHSS and a strength assessment, using the Medical Research Council scale to rate the deltoid, biceps, triceps, wrist extension, wrist flexion, intrinsic finger, hip flexor, quadriceps, hamstrings, ankle extension, and ankle flexion ranging from 0 (no movement) to 5 (full strength) on each side.

### Monitoring

The subjects had wrist straps incorporating accelerometers placed on both arms. For the algorithm derivation cohort we used a commercially available battery-powered Bluetooth-enabled accelerometer/gyroscope, the Wit Motion (Shenzhen City, China) BWT901 CL Bluetooth output 9-axis accelerometer gyroscope, synced with an Android tablet to stream the data to a cloud-based server (Heroku, Inc., San Francisco, CA). The accelerometry devices had an expected battery life of 2-3 hours. To capture more data and allow for comparisons of performance between daytime and nighttime, we required a longer lasting accelerometry device. Thus, for the validation cohort, we used the commercially available Samsung Galaxy Watch Active to collect accelerometry data. An app collected accelerometry data (Raproto, Philadelphia, PA) which was transmitted via WiFi to a cloud-based platform for storage (Thingsboard, Inc, New York, NY).²⁰ The expected battery life of this device was 18-24 hours. For both phases of the study, patients and clinical staff were told that the straps could be removed at any time if they were uncomfortable, interfered with clinical treatment, or for any other reason they chose. To ensure conditions were representative of real-world practice, no instructions to limit therapy or passive range motion of the affected limb were given while the patient was being monitored. The neurological assessments were repeated after monitoring was complete to confirm that there were no changes in neurologic status.

### Algorithm Derivation

The algorithm was derived using a parameter-invariant (PAIN) method designed to maximize diagnostic performance and generalizability.²¹⁻²⁵ This approach has been previously used to develop multiple medical classifier algorithms requiring high sensitivity and specificity along with stable performance across patients without outliers.²² The PAIN method uses a statistical first-principle approach to derive algorithms that are invariant to patient-specific parameters (e.g., being left- or right-handed, awake/asleep, restrained/free-to-move) as well as system anomalies common in accelerometer-based systems (e.g., accelerometer bias/drift or device orientation). As a result, the algorithm achieves stable performance across the population without requiring individual tuning.

The algorithm derivation methodology is described further below. Briefly, utilizing the derivation cohort accelerometry data we identified features invariant to patient-specific parameters and then trained a structured classification tree combining the features to maximize stability and accuracy for detection of asymmetric movement patterns seen in patients with stroke. Using multiple concurrent threshold tests of varying durations can balance the trade-off between accuracy and time-to-detection.²⁶ Threshold tests with shorter monitoring durations provide faster time-to-detection while longer monitoring durations have increased accuracy. The algorithm simultaneously utilizes multiple windows of increasing duration of preceding data (when available) and alarms if any window detects the possible presence of a stroke.

In some examples, an alarm that leads to identification of a stroke triggers a clinical intervention that would include removing the device. Thus, if movement data continue to accrue after an alarm, the algorithm assumes that the prior alarm was a false positive and no further alarms are generated for 1 hour to allow the monitoring windows to accumulate new data. Every subsequent alarm within 4 hours of the previous alarm extends the alarm pause by an additional hour up to a maximum of 4 hours. If there is no generated alarm within 8 hours, the alarm pause duration is reset to 1 hour. We note that the proposed strategy results in a maximum false alarm rate of 8 alarms in the first 24 hours followed by 6 alarms per day from then on.

### Validation

A candidate algorithm was validated using an independent and blinded test dataset that was collected separately from the dataset used for algorithm derivation using a different, longer lasting accelerometer as noted above.²⁷ For this analysis, the algorithm evaluated individual patient data and was executed every 15 minutes. For control subjects without stroke, we evaluated the algorithm performance in terms of false alarms per patient per day, defined as the number of alarms divided by the monitoring time in days. We report the median false alarm per day by taking the median of the false alarm per day over all control subjects. For each case subject with stroke, we evaluated the algorithm performance in terms of detection rate as time from initiation of monitoring increased. Start times of monitoring were in 15-minute increments throughout the entire duration of monitoring for each patient. Since data for subjects who transition from neurologically intact to having a stroke during monitoring were not available in our study, we utilized a conservative evaluation for detection rate versus time-to-detection commonly employed in the quickest detection literature.²⁸ As the time from initiation of monitoring increases, the aggregate test includes only windows of shorter duration and the detection rate is calculated based on the percentage of aggregate tests that identified stroke.

As noted above, a false alarm will lead to a transient pause in alarm generation. In order to account for how this feature impacts the time to detection in stroke cases, we calculated the duration that the alarm was paused per day based on the algorithm performance in the control subjects. The median and interquartile range of the delay due to pauses was then added to time to detection for the stroke cases. For example, if the median false alarm rate in controls was 1 per day, the alarm would be paused for 1 hour out of 24 hours. Assuming that a stroke can occur at any time during the 24 hour period, there will be 23 hours with no additional delay and 1 hour when the alarm is paused (with a median delay of 30 minutes), ( 23/24)*0 minutes+( 1/24)*30 minutes=1.25 minutes additional expected delay per day.²⁷ Finally, we evaluated whether patient specific factors would lead to variations in performance of the algorithm by comparing the median time to detection and false alarm rates by handedness, non-dominant hemisphere involvement, and whether monitoring occurred during nighttime or daytime using Wilcoxon ranksum testing.

### Results

From May 8, 2018 through Nov. 23, 2021 we enrolled 405 patients including 200 in the derivation cohort and 205 in the validation cohort. Accelerometry data were not available for 5 control subjects in the validation cohort due to technical difficulties and they were excluded from the analysis. The algorithm derivation cohort included 77 patients with acute stroke and lateralizing arm weakness and 123 neurologically intact control subjects. In total, 540 hours of bilateral arm accelerometry data was acquired during this phase. The algorithm validation cohort included 33 patients with acute stroke and 167 controls totaling 4169 hours of bilateral arm accelerometry data. Table 1 presents the clinical and demographic characteristics of the controls for the derivation and validation cohorts and Table 2 provides these data for the stroke cases. Among the non-stroke controls, subjects in the validation cohort were less likely to be female and were more likely to have recently had surgery. For stroke cases, the validation cohort was similar to the derivation cohort with the exception of a greater difference in arm strength between the affected and unaffected side, as measured by the sum of the medical research council upper extremity motor scores, although the differential in the NIHSS upper extremity motor score was similar.

Within the validation cohort, stroke cases had similar age (mean 68 vs 65 years. P=0.25), percentage of females (45% vs 32%, p=0.15), and percentage who were right-handed (94% vs 84%, p=0.13) compared to the controls, but stroke patients were more often non-white (45% vs 11%, p<0.001) and were more often in an ICU or step-down unit (85% vs 34%, p<0.001). Overall, stroke cases in the validation cohort were predominantly ischemic (73%) and moderately severe (NIHSS median 14, IQR 9-18). For both the algorithm derivation and validation cohorts, the wrist straps were well tolerated. None of the patients in the derivation cohort and two patients in the validation cohort removed the devices and prematurely terminated the study (after 1 hour and 22 hours of monitoring, respectively). Nurses reported no issues with the straps interfering with clinical care. There were no changes in patient upper extremity strength or presence of neglect comparing the examinations at baseline and study completion.

TABLE 1
Clinical and demographic characteristics of neurologically normal
controls in the algorithm derivation and validation cohorts

| | | | Derivation | Validation | |
|---|---|---|---|---|---|
| | Total | | cohort | cohort | P- |
| | (n = 290) | | (n = 123) | (n = 167) | value |
| Age in years, | 64 ± 15 | | 62 ± 18 | 65 ± 12 | 0.06 |
| mean ± standard | | | | | |
| deviation | | | | | |
| Female sex | 109 | (38%) | 55 (45%) | 54 (32%) | 0.03 |
| Nonwhite race | 42 | (14%) | 23 (19%) | 19 (11%) | 0.08 |
| Left-handed | 39 | (13%) | 12 (10%) | 27 (16%) | 0.13 |
| Admission reason | | | | | <0.001 |
| TIA | 1 | (0.3%) | 1 (1%) | 0 | |
| Epilepsy | 24 | (8%) | 24 (19%) | 0 | |
| monitoring | | | | | |
| Surgery | 265 | (91%) | 98 (80%) | 167 (100%) | |

| | | | | |
|---|---|---|---|---|
| Monitoring duration | 972 (174- | 171 (135- | 1320 (1216- | <0.001 |
| in minutes, median | 1340) | 190) | 1404) | |
| (interquartile range) | | | | |

Continuous variables presented as median (interquartile range) unless otherwise specified

TABLE 2
Clinical and demographic characteristics of stroke
cases in the derivation and validation cohorts

| | | Derivation | | Validation | | |
|---|---|---|---|---|---|---|
| | Total | cohort | | cohort | | |
| | (n = 110) | (n = 77) | | (n = 33) | | P-value |
| Age in years, mean ± | 68 ± 16 | 68 ± 15 | | 68 ± 17 | | 0.95 |
| standard deviation | | | | | | |
| Female sex | 53 (48%) | 38 | (49%) | 15 | (45%) | 0.71 |
| Nonwhite race | 51 (46%) | 36 | (47%) | 15 | (45%) | 0.90 |
| Left-handed | 11 (10%) | 9 | (12%) | 2 | (6%) | 0.33 |
| Stroke type | | | | | | 0.10 |
| Intracerebral | 20 (18%) | 11 | (14%) | 9 | (28%) | |
| hemorrhage | | | | | | |
| Ischemic stroke | 90 (82%) | 66 | (86%) | 24 | (73%) | |
| Non-dominant | 40 (36%) | 27 | (35%) | 13 | (39%) | 0.67 |
| hemispheric stroke | | | | | | |
| Total NIHSS Score at | 13 (8- | 12 (7- | | 14 (9- | | 0.16 |
| time of monitoring | 18) | 16) | | 18) | | |
| Difference in NIHSS | 4 (3- | 4 (2- | | 4 (3- | | 0.22 |
| upper extremity motor | 4) | 4) | | 4) | | |
| score between | | | | | | |
| affected and | | | | | | |
| unaffected side | | | | | | |
| Difference in sum of | 24 (18- | 24 (16- | | 30 (22- | | 0.01 |
| upper extremity | 30) | 30) | | 30) | | |
| strength scores | | | | | | |
| between affected and | | | | | | |
| unaffected side* | | | | | | |
| Weakness from | 66 (60%) | 47 | (61%) | 19 | (58%) | 0.73 |
| stroke on left side | | | | | | |
| Neglect present | 54 (49%) | 36 | (47%) | 18 | (55%) | 0.45 |

| | | | | |
|---|---|---|---|---|
| Monitoring duration | 190 (168- | 178(150- | 1299 (1235- | <0.001 |
| in minutes | 1102) | 192) | 1408) | |

Continuous variables presented as median (interquartile range) unless otherwise specified
*Upper extremity muscle groups assessed with the Medical Research Council muscle strength score (ranging from 0-5) included deltoid, biceps, triceps, wrist extension, wrist flexion, and intrinsic finger strength with full strength in all 6 muscles tested scoring a 30.

### Algorithm Performance

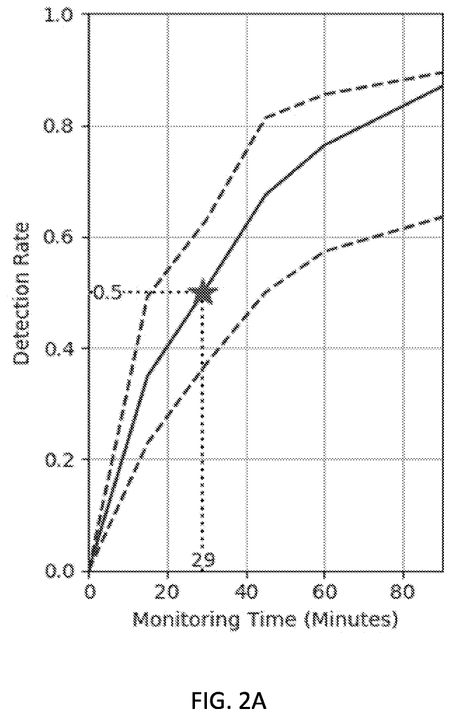

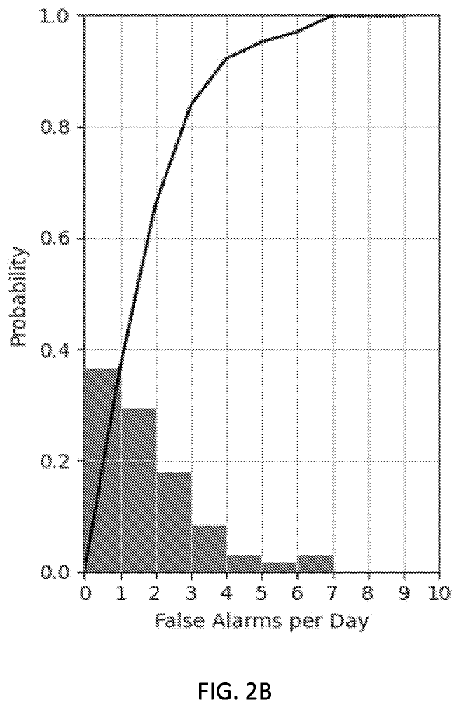

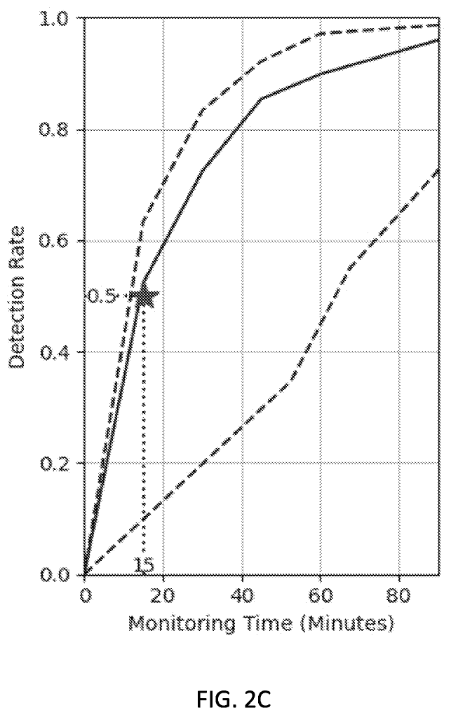

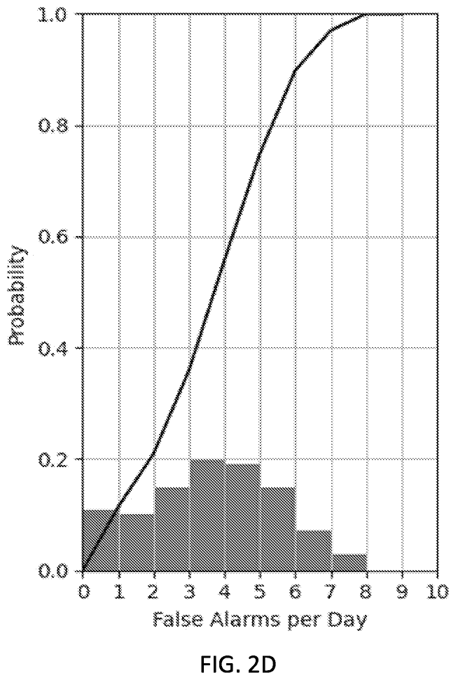

FIGS. 2A-2D display the median and interquartile range (IQR) for the percentage of stroke cases that alarm as monitoring time increases using two different alarm thresholds. FIGS. 2A-2D show stroke detection rate over time and false alarm rates per day.

FIG. 2A shows the median (solid line) and interquartile range (dashed lines) of the percentage of patients with stroke alarming as duration of monitoring increases. FIG. 2B shows the distribution of false alarms per patient per day in non-stroke controls. The black line represents the cumulative percentage of patients. FIG. 2C shows the impact of a lower alarm threshold on time to detection. FIG. 2D shows the impact of a lower alarm threshold on false alarm rates.

The sensitivity (i.e., the percentage of stroke patients detected as having a stroke) was positively correlated with the duration of monitoring. Comparing the results from the two different target false alarm rates demonstrates that the sensitivity and false alarm rate were also correlated.

As false alarm rates increase, the times to detection decrease. With a median false alarm rate among non-stroke controls of 1.1 alarms per patient per day (IQR 0 to 2.2 alarms per patient per day), the median time to alarm in stroke cases was 29 minutes (IQR 11 to 58 minutes). At 60 minutes, the algorithm is expected to detect 76% of strokes. With a median false alarm rate of 3.6 alarms per patient per day (IQR 2.1 to 5.0 false alarms), the median time to detection in stroke cases was 15 minutes (IQR 8 to 74 minutes). At this setting, the algorithm is expected to detect 91% of strokes at 60 minutes. Importantly, the algorithm was unaffected by patient-specific factors that could theoretically lead to variable performance. Specifically, using the lower sensitivity threshold, there was no significant difference in false alarm rates (median 1.2 vs 1.0 alarms per day, p=0.17) or time to detection (median 29 vs 27 minutes, p=0.83) comparing right-handed vs left-handed patients. There was similarly no difference in time to detection (median 29 vs 29 minutes, p=1.0) if the stroke involved the dominant or non-dominant hemisphere. Most importantly, there were no differences in false alarms detected (median 0 vs 0 alarms, p=0.57) or time to detection (median 28 vs 26 minutes, p=0.79) comparing daytime vs nighttime. These results were similar when evaluated using the threshold with increased sensitivity.

### Discussion

This study demonstrates that arm accelerometry data can be used to discriminate patients with weakness caused by acute stroke from neurologically intact hospitalized patients. The algorithm's diagnostic performance achieves a high sensitivity and specificity such that it could provide a clinically useful monitor to rapidly detect the onset of stroke while maintaining a low false alarm rate. The alarm threshold is modifiable and a lower threshold demonstrated greater sensitivity and faster time to detection, with a concomitant higher false alarm rate. Importantly, the estimate of the time to detection is conservative and may be faster in clinical use. In the analyses of time to detection, we only included movement data from patients with stroke.

In practice, patients will convert from non-stroke to stroke in the midst of an evaluation window, which may still trigger an alarm, yielding faster times from onset to detection than we report. In addition, stroke cases were cared for in real-world routine clinical practice while they were being monitored and there were times when the care team or family members would move the patients' weak arms. These time periods were not censored for the validation analysis. In clinical use, we expect that the algorithm will detect over half of strokes within 30 minutes of onset, while maintaining well less than two false alarms per day for the vast majority of patients. Of greatest importance, we saw no significant variability in algorithm performance based on handedness, non-dominant hemispheric involvement, or whether we were monitoring during daytime vs nighttime. This latter finding suggests that we can detect stroke equally during sleep or wakefulness, which is a critical feature of a useful stroke monitor.

In-hospital stroke is a major public health issue which accounts for a meaningful portion of all strokes and is associated with delayed assessment and treatment, poor outcome, and dramatically increased cost and length of stay.⁷⁻¹² Importantly, periprocedural stroke accounts for the majority of cases in most series and stroke rates for common procedures such as aortic valve surgery are much higher than commonly reported when prospective assessments are performed.^{7-9,29-31} Given that the algorithm detects asymmetry and is not based on change in movement patterns from a baseline period, it is particularly well suited to detect stroke in the perioperative setting where patients may awake from anesthesia with weakness. Prior studies of in-hospital stroke have reported times from last known normal to symptom detection ranging from −2 to 10 hours.²⁹⁻³¹ While proven stroke treatments may have robust benefit, the likelihood of being able to receive these treatments and the response to treatment steadily decline over time.¹⁻³ Thus, rapid detection of the onset of stroke remains critically important. A device incorporating this algorithm to continuously monitor for stroke onset could dramatically reduce the time to assessment, leading to more and faster interventions and better outcomes for patients.

Upper limb weakness is one of the most common symptoms of acute stroke, seen in ˜75% of patients.¹⁵ For this reason, pre-hospital stroke screening tools and scales that aim to identify patients with the greatest likelihood of having a large vessel occlusion have all included arm strength.^{16,17,32} In addition, attentional neglect is present in 20-70% of strokes and studies of stroke patients using wrist-worn accelerometers have demonstrated that neglect is associated with asymmetric movement.^{18,19} Importantly, weakness and neglect are both strongly associated with long term disability from stroke.^{33,34} Thus, while upper limb accelerometry monitoring will not capture every stroke, it will identify the vast majority of strokes including those most likely to result in disability and be most amenable to thrombectomy, which is proven to dramatically improve outcomes.

Patient physiologic monitors are ubiquitous in hospitals in general and in intensive care units in particular, where multimodal monitoring is standard of care. Unfortunately, these pervasive monitors may result in alarm fatigue leading to delayed or absent responses.³⁵ Fatigue is more likely when non-actionable alarms are much more prevalent than actionable alarms that require both clinical awareness and intervention. Stroke is a critical patient event that is both actionable and exquisitely time sensitive.⁶ A study of 461 adults treated in intensive care units annotated a total of 381,560 unique audible alarms over a 31-day study period.³⁶ Accelerated ventricular arrhythmia alarms, a potentially critical patient abnormality, occurred at an average of 4.5 alarms per patient per day of monitoring of which only 12 (0.3%) were clinically relevant actionable events. This stroke detection algorithm provides a far lower false alarm rate, while greatly reducing time from symptom onset to stroke detection compared to current clinical practice.

This study demonstrates that arm accelerometry data can differentiate patients with acute stroke from neurologically normal hospitalized patients at risk of stroke. Notably, we performed the validation analysis on a separate prospectively acquired cohort of patients, using different accelerometry devices than were used to collect data to derive the algorithm. The performance of the algorithm under these conditions reflects its robustness and generalizability. The validation cohort included control patients who underwent cardiothoracic or vascular surgical procedures reflecting a population that is high risk for stroke and would benefit from continuous stroke monitoring.

### CONCLUSIONS

In-hospital stroke is a major public health issue and a monitor that can rapidly detect the onset of stroke and facilitate expedited assessment and treatment would lead to greatly improved outcomes for patients. We derived a stroke detection algorithm using upper extremity accelerometry data from hospitalized patients that demonstrates promising diagnostic performance in a prospective validation cohort. A trial to prospectively monitor patients at risk of stroke is required to demonstrate clinical utility and tolerability.

### REFERENCES

1. Saver J L. Time is brain—quantified. Stroke. 2006; 37:263-266
2. Goyal M, Menon B K, van Zwam W H, Dippel D W, Mitchell P J, Demchuk A M, Davalos A, Majoie C B, van der Lugt A, de Miquel M A, et al. Endovascular thrombectomy after large-vessel ischaemic stroke: A meta-analysis of individual patient data from five randomised trials. Lancet. 2016; 387:1723-1731
3. Saver J L, Goyal M, van der Lugt A, Menon B K, Majoie C B, Dippel D W, Campbell B C, Nogueira R G, Demchuk A M, Tomasello A, et al. Time to treatment with endovascular thrombectomy and outcomes from ischemic stroke: A meta-analysis. JAMA. 2016; 316:1279-1288
4. Alberts M J, Perry A, Dawson D V, Bertels C. Effects of public and professional education on reducing the delay in presentation and referral of stroke patients. Stroke. 1992; 23:352-356
5. Lecouturier J, Rodgers H, Murtagh M J, White M, Ford G A, Thomson R G. Systematic review of mass media interventions designed to improve public recognition of stroke symptoms, emergency response and early treatment. BMC Public Health. 2010; 10:784
6. Powers W J, Rabinstein A A, Ackerson T, Adeoye O M, Bambakidis N C, Becker K, Biller J, Brown M, Demaerschalk B M, Hoh B, et al. Guidelines for the early management of patients with acute ischemic stroke: 2019 update to the 2018 guidelines for the early management of acute ischemic stroke: A guideline for healthcare professionals from the american heart association/american stroke association. Stroke. 2019; 50:e344-e418
7. Dulli D, Samaniego E A. Inpatient and community ischemic strokes in a university hospital. Neuroepidemiology. 2007; 28:86-92
8. Park H J, Cho H J, Kim Y D, Lee D W, Choi H Y, Kim S M, Heo J H. Comparison of the characteristics for in-hospital and out-of-hospital ischaemic strokes. Eur J Neurol. 2009; 16:582-588
9. Saltman A P, Silver F L, Fang J, Stamplecoski M, Kapral M K. Care and outcomes of patients with in-hospital stroke. JAMA Neurol. 2015; 72:749-755
10. Cumbler E, Wald H, Bhatt D L, Cox M, Xian Y, Reeves M, Smith E E, Schwamm L, Fonarow G C. Quality of care and outcomes for in-hospital ischemic stroke: Findings from the national get with the guidelines-stroke. Stroke. 2014; 45:231-238
11. Nouh A, Amin-Hanjani S, Furie K L, Kernan W N, Olson D M, Testai F D, Alberts M J, Hussain M A, Cumbler E U, American Heart Association Stroke C, et al. Identifying best practices to improve evaluation and management of in-hospital stroke: A scientific statement from the american heart association. Stroke. 2022:STR0000000000000402
12. Puskas J D, Winston A D, Wright C E, Gott J P, Brown W M, 3rd, Craver J M, Jones E L, Guyton R A, Weintraub W S. Stroke after coronary artery operation: Incidence, correlates, outcome, and cost. Ann Thorac Surg. 2000; 69:1053-1056
13. Bhatt A, Safdar A, Chaudhari D, Clark D, Pollak A, Majid A, Kassab M. Medicolegal considerations with intravenous tissue plasminogen activator in stroke: A systematic review. Stroke Res Treat. 2013; 2013:562564
14. Haslett J J, Genadry L, Zhang X, LaBelle L A, Bederson J, Mocco J, Kellner C P. Systematic review of malpractice litigation in the diagnosis and treatment of acute stroke. Stroke. 2019; 50:2858-2864
15. Lawrence E S, Coshall C, Dundas R, Stewart J, Rudd A G, Howard R, Wolfe C D. Estimates of the prevalence of acute stroke impairments and disability in a multiethnic population. Stroke. 2001; 32:1279-1284
16. van Gaal S C, Kamal N, Betzner M J, Vilneff R L, Mann B, Lang E S, Demchuk A, Buck B, Jeerakathil T, Hill M D. Approaches to the field recognition of potential thrombectomy candidates. Int J Stroke. 2017; 12:698-707
17. Duvekot M H C, Venema E, Rozeman A D, Moudrous W, Vermeij F H, Biekart M, Lingsma H F, Maasland L, Wijnhoud A D, Mulder L, et al. Comparison of eight prehospital stroke scales to detect intracranial large-vessel occlusion in suspected stroke (presto): A prospective observational study. Lancet Neurol. 2021; 20:213-221
18. Chen P, Hreha K, Fortis P, Goedert K M, Barrett A M. Functional assessment of spatial neglect: A review of the catherine bergego scale and an introduction of the kessler foundation neglect assessment process. Top Stroke Rehabil. 2012; 19:423-435
19. Trejo-Gabriel-Galan J M, Rogel-Melgosa V, Gonzalez S, Sedano J, Villar J R, Arenaza-Basterrechea N. Rehabilitation of hemineglect of the left arm using movement detection bracelets activating a visual and acoustic alarm. J Neuroeng Rehabil. 2016; 13:79
20. Watson A, Choi H, Lee I, Weimer J. Raproto: An open source platform for rapid prototyping of wearable medical devices. Proceedings of the Workshop on Medical Cyber Physical Systems and Internet of Medical Things. 2021:1-6
21. Ivanov R, Weimer J, Simpao A, Rehman M, Lee I. Early detection of critical pulmonary shunts in infants. Proceedings of the ACM/IEEE Sixth International Conference on Cyber-Physical Systems. 2015:110-119
22. Weimer J, Chen S, Peleckis A, Rickels M R, Lee I. Physiology-invariant meal detection for type 1 diabetes. Diabetes Technol Ther. 2016; 18:616-624
23. Weimer J, Ivanov R, Chen S, Roederer A, Sokolsky O, Lee I. Parameter-invariant monitor design for cyber-physical systems. Proceedings of the IEEE. 2017:71-92
24. Nguyen H, Jang S, Ivanov R, Bonafide C P, Weimer J, Lee I. Reducing pulse oximetry false alarms without missing life-threatening events. Smart Health (Amst). 2018; 9-10:287-296
25. Weimer J, Ivanov R, Roederer A, Chen S, Lee I. Parameter-invariant design of medical alarms. IEEE Design & Test. 2015; 32:9-16
26. Wilsky A. A survey of design methods for failure detection in dynamic systems. Automatica. 1976; 12:601-611
27. Bishop C. Pattern recognition and machine learning. New York: Springer; 2006.
28. Poor H, Hadjiliadis O. Quickest detection. Cambridge, U K: Cambridge University Press; 2008.
29. Kassardjian C D, Willems J D, Skrabka K, Nisenbaum R, Barnaby J, Kostyrko P, Selchen D, Saposnik G. In-patient code stroke: A quality improvement strategy to overcome knowledge-to-action gaps in response time. Stroke. 2017; 48:2176-2183
30. Manners J, Khandker N, Barron A, Aziz Y, Desai S M, Morrow B, Delfyett W T, Martin-Gill C, Shutter L, Jovin T G, et al. An interdisciplinary approach to inhospital stroke improves stroke detection and treatment time. J Neurointerv Surg. 2019; 11:1080-1084
31. Cummings S, Kasner S E, Mullen M, Olsen A, McGarvey M, Weimer J, Jackson B, Desai N, Acker M, Messe S R. Delays in the identification and assessment of in-hospital stroke patients. J Stroke Cerebrovasc Dis. 2022; 31:106327
32. Brandler E S, Sharma M, Sinert R H, Levine S R. Prehospital stroke scales in urban environments: A systematic review. Neurology. 2014; 82:2241-2249
33. Kwakkel G, Wagenaar R C, Kollen B J, Lankhorst G J. Predicting disability in stroke—a critical review of the literature. Age Ageing. 1996; 25:479-489
34. Cherney L R, Halper A S, Kwasnica C M, Harvey R L, Zhang M. Recovery of functional status after right hemisphere stroke: Relationship with unilateral neglect. Arch Phys Med Rehabil. 2001; 82:322-328
35. Hravnak M, Pellathy T, Chen L, Dubrawski A, Wertz A, Clermont G, Pinsky M R. A call to alarms: Current state and future directions in the battle against alarm fatigue. J Electrocardiol. 2018; 51:S44-S48
36. Drew B J, Harris P, Zegre-Hemsey J K, Mammone T, Schindler D, Salas-Boni R, Bai Y, Tinoco A, Ding Q, Hu X. Insights into the problem of alarm fatigue with physiologic monitor devices: A comprehensive observational study of consecutive intensive care unit patients. PLoS One. 2014; 9:e110274

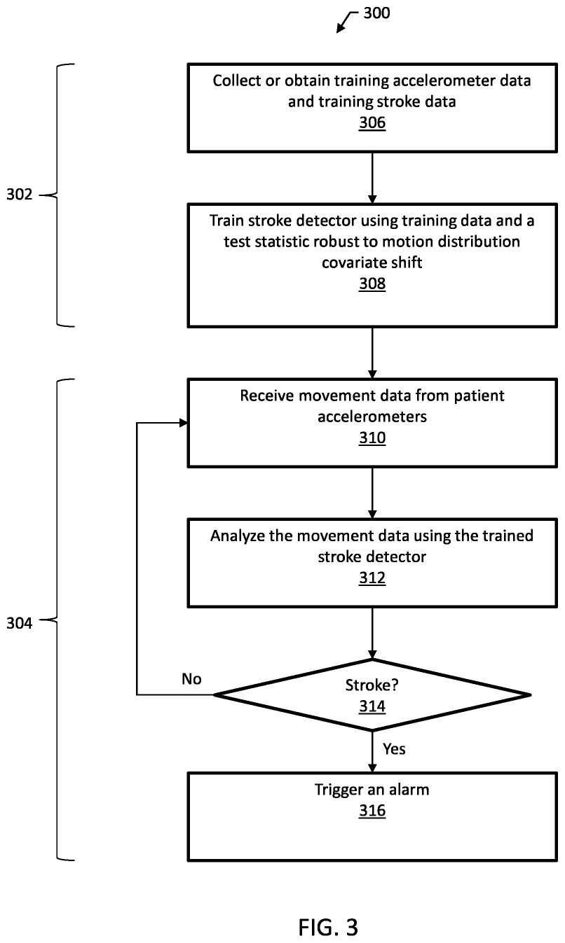

FIG. 3 is a flow diagram illustrating an example method 300 for detecting stroke. The method 300 includes a training phase 302 and a detection phase 304. Typically, the training phase 302 is performed first by a first computer system to produce a stroke detector, i.e., a model comprising data to be distributed to other computer systems. Other computer systems can then individually perform the detection phase 304, where the model is used to detect stroke in patients.

The method 300 includes collecting or obtaining training data (306). The training data includes accelerometer data obtained from patients and stroke data indicating whether corresponding accelerometer data was taken from a patient experience a stroke or not.

The method 300 includes training a stroke detector using the training data and a test statistic robust to motion distribution covariate shift (308). As a result of training the stroke detector, a model is produced. The model comprises data that can be stored on individual computer systems or in cloud systems, e.g., as described above with reference to FIGS. 1A and 1B. Training the stroke detector is described further below with respect to an example.

The method 300 includes receiving movement data from one or more patient accelerometers (310). In some examples, receiving movement data includes receiving the movement data from wireless signals from wrist-mounted accelerometers on both of a patient's wrists. Receiving the movement data can include pre-processing the movement data to remove the effect of rotation/sliding of the accelerometer and bias. Although accelerometry data alone may be sufficient for stroke detection, in some examples, other data is collected, e.g., data from other sensors for detecting motion such as gyroscopes and magnetometers.

The method 300 includes analyzing the movement data using the trained stroke detector (312) and determining whether or not the patient is experiencing a stroke (314). If the stroke detector determines that the patient is experiencing a stroke, then method 300 includes triggering an alarm to a caregiver (316). Otherwise, the method 300 continues to receive movement data (return to 310) until a stroke is detected or patient monitoring is ended.

### Stroke Detector Training Example

### Data Pre-Processing

To design a low-cost lightweight comfortable wrist-worn device for stroke detection, we sought to utilize only accelerometry data in our stroke detection analysis. While incorporating additional sensors, such as gyroscopes and magnetometers, would theoretically enable device orientation and arm position estimation, they would also increase device cost, power consumption, battery size, and weight. Consequently, this work aimed to utilize off-the shelf low-power accelerometers to detect stroke and the pre-processing considered herein assumed only accelerometry data were available.

Low-cost low-power accelerometers common in wrist-worn devices produced, at time k, 3-dimensional data, aₓ(k), a_{y}(k), and a_{z}(k), but were also susceptible to bias and rotation/sliding on the wrist. We denoted the constant bias as cₓ, c_{y}, and c_{z}, and removed their effect by utilizing the first-derivative of acceleration (known as “jerk”) since

$${J}_{x} ( k ) = \frac{d}{dk} ( {a}_{x} ( k ) + {c}_{x} ) = \frac{d}{dk} {a}_{x} ( k ) ,$$

and similarly for the y and z dimensions. Once the bias was removed, we removed the effect of rotation/sliding on the wrist by only considering the magnitude of jerk, written for the left-arm motion data as x_{L}(k)=\sqrt{J_{x,L}²(k)+J_{y,L}²(k)+J_{z,L}²(k)}∈X, where X denotes the feature space and a similar equation exists for the right arm, x_{R}(k). This pre-processing step served to eliminate inherent system biases that are likely to occur during real-world deployments and are consistent with other data pre-processing techniques for accelerometry data without access to gyroscopes and magnetometers.

### Test Statistic Engineering

To engineer a test statistic for discriminating the between stroke and neurologically intact subjects, we began by writing D_{X}={f:X→P}, to be a space of probability distributions mapping the feature space to a probability. In an (idealized) controlled evaluation environment, where a subject performs a prescribed sequence of actions/motions, the distribution for the left arm, f_{L}∈D_{X}, and right arm, f_{R}∈D_{X}, can discriminate between neurologically intact subjects (i.e., f_{L} f_{R}) and stroke subjects (i.e., f_{L}≠f_{R}). While this idealized scenario can yield highly sensitive and specific stroke detection, in practice it would be far too invasive—requiring frequent neurological assessments to timely detect stroke.

Rather than require patients to perform a set of prescribed tasks at set intervals, we sought to engineer a test statistic that is suitable for passive monitoring scenarios. Such a test statistic must be robust to changes in the underlying patient motion distribution, referred to in the statistical literature as a covariate shift.¹ Motion distribution covariate shift is common in passive monitoring scenarios and captures the effect of any patient-specific tendency in the data (e.g., dominant hand, comorbidities, etc.). However, the impact of the motion covariate shift will be limited by the patient's neurological state, which is presumed to be unknown at the time of testing. Consequently, we modeled the family of motion covariate shifts as a group of distribution nuisance transformations applied to the patient's (unknown) neurological state, namely for f∈{f_{L},f_{R}},

$${G}_{f} = \{ g : {D}_{X} \to {D}_{X} \vert ∀ x ∈ X , f ( x ) \neq 0 ↔ g ( f ( x ) ) \neq 0 \}$$

where g∈G_{f} denotes a potential motion covariate shift. Consequently, we seek a test statistic that can assess the neurological state robust to motion distribution covariate shift.

One example approach to realize a robust test statistic utilizes parameter invariant (PAIN) statistics—which have been previously applied in multiple domains.²⁻⁵ Given a group of nuisance transformations, a PAIN statistic, t, seeks to provide invariance to the nuisance transformations (i.e., is invariant: ∀f∈D_{X}, ∀g∈G,t(g(f))=t(f)) while only eliminating information affected by the nuisance transformations, (i.e., is maximal ∀f, f′∈ D_{X}, [symbol] g∈G, t(f)=t(f′)→g(f)=f′). Thus, we considered a candidate PAIN statistic,

$$t : d ∈ {D}_{X} ↦ {d}^{′} ∈ {D}_{X} : ∃ c , ∀ x , ∈ X , c 1 ( d ( x ) \neq 0 ) \\ and proved it to be invariant since , ∀ f ∈ {D}_{X} , ∀ g ∈ G \\ ∀ g ∈ G , t ( g ( f ) ) = {d}^{′} ∈ {D}_{X} : ∃ x , ∀ x ∈ X , c 1 ( g ( f ( x ) ) \neq 0 ) = {d}^{′} ∈ {D}_{X} : ∃ c , ∀ x ∈ X , c 1 ( f ( x ) ) \neq 0 ) = t ( f ) \\ and maximal since , ∀ f , {f}^{′} ∈ {D}_{X} , ∃ g ∈ G , \\ t ( f ) = t ( {f}^{′} ) \to d ∈ {D}_{X} : ∃ c , ∀ x ∈ X , c 1 ( f ( x ) \neq 0 ) = {d}^{′} ∈ {D}_{X} : ∃ {c}^{′} , ∀ x ∈ X , {c}^{′} 1 ( {f}^{′} ( x ) \neq 0 ) \to g ( f ) = {f}^{′}$$

Moreover, we note that t(f_{L}) and t(f_{R}) have an attractive property, namely if f_{L} f_{R} (as is the case in neurologically intact subjects in the idealized scenario), then t(f_{L}) t(f_{R}), stated formally as f_{L}=f_{R}→t(f_{L}) t(f_{R}). This means that in the idealized monitoring scenario, if subjects are neurologically intact, then in the passive monitoring scenario they should also appear neurologically intact.

Thus, we aimed to generate a test statistic, that discriminated between neurologically intact subjects (i.e., t(f_{L})=t(f_{R})) and stroke subjects (i.e., t(f_{L})≠t(f_{R})). In this scenario, we utilized the Kolmogorov-Smirnov (KS) statistic,^{6,7} denoted by letting t_{L}=t(f_{L}) and t_{R}=t(f_{R}), and writing the test statistic

$$s = {sup}_{z ∈ X} \vert {\int }_{- ∞}^{Z} {t}_{L} ( x ) - {t}_{R} ( x ) dx \vert$$

which, represents a non-parametric statistic of distribution equality that equals the maximum absolute deviation of the cumulative distribution functions corresponding to the probability mass functions t_{L} and t_{R}. The KS statistic is a widely used test of distribution equality when the underlying test distribution family is unknown or non-parameterized (i.e., non-parametric).

### Test Generation

We then developed a threshold test for the test statistic, s, derived in the previous section. The test statistic requires the cumulative distribution functions corresponding to the probability mass functions t_{L} and t_{R}. Unfortunately, these are not generally known and must be estimated from a recent history (1 hour) of the pre-processed sampled data, X(k)={(x_{L}(k),x_{R}(k)),(x_{L}(k−1),x_{R}(k−2)), . . . }. Utilizing sampled data estimates in place of the actual distribution presents two potential concerns. First, when there is significant missing data the amount of information contained in the sampled data decreases. Second, anytime the patient has no motion (i.e., laying perfectly still) while the data is not technically missing, it provides no discriminatory information for testing stroke versus neurologically intact. Consequently, we write s(k) to be the test statistic estimated using X(k), and write r₁(k)=|X(k)| to be the number of data points in X(k) and r₂(k)=|{(X_{L},X_{R})|(X_{L},X_{R})∈X(k),x_{L}≠0Vx_{R}≠0}|/|X(k)| to be the percentage of X(k) with patient movement.

To derive a threshold test we leveraged r₁ and r₂ to adapt a threshold such that the resulting test has a constant false alarm rate, α∈[0,1]. To achieve this, we grouped the data using kmeans with k=100 on [(r₁(1),r₂(1)), (r₁(2),r₂(2)), . . . ] and generated a corresponding threshold for each group to achieve a constant false alarm rate α. To achieve maximal distributional accuracy when tuning the false alarm rate the threshold test was calibrated prior to threshold selection.⁸ At runtime, a new s(k) was generated with corresponding r₁(k) and r₂(k). The decision threshold utilized for testing s(k) corresponds to the group containing (r₁(k),r₂(k)). In the following, we refer to the threshold test described above as ϕ∈{0,1}, where ϕ=0 predicts the absence of stroke and ϕ=1 predicts the presence of stroke.

To improve sensitivity to the onset of stroke, we ran multiple threshold tests, ϕ₁, . . . , ϕ_{L}, simultaneously with different monitoring durations, d₁, . . . , d_{L}, respectively. For example, for each l∈{1, . . . ,L} at time t, ϕ₁ utilized data in the time range [t−d₁,t]. Leveraging the multiple threshold tests, we defined an aggregate threshold test, \hat{ϕ}=max{ϕ₁, . . . , ϕ_{L}}, that predicts the presence of stroke if and only if one of the L monitoring durations predicts the presence of a stroke. We note that the false alarm rate of the aggregate test is always greater than α. Consequently, we select α in the threshold test design to be small enough such that the aggregate test achieves our desired false alarm rate.

### REFERENCES

1. McGaughey G, Walters W P, Goldman B. Understanding covariate shift in model performance. F1000Research. 2016; 5
2. Ivanov R, Weimer J, Simpao A, Rehman M, Lee I. Early detection of critical pulmonary shunts in infants. Proceedings of the ACM/IEEE Sixth International Conference on Cyber-Physical Systems. 2015:110-119
3. Weimer J, Chen S, Peleckis A, Rickels M R, Lee I. Physiology-invariant meal detection for type 1 diabetes. Diabetes technology & therapeutics. 2016; 18:616-624
4. Weimer J, Ivanov R, Chen S, Roederer A, Sokolsky O, Lee I. Parameter-invariant monitor design for cyber-physical systems. Proceedings of the IEEE. 2017:71-92
5. Nguyen H, Jang S, Ivanov R, Bonafide C P, Weimer J, Lee I. Reducing pulse oximetry false alarms without missing life-threatening events. Smart health. 2018; 9-10:287-296
6. Kolomogorov A. Sulla determinazione empirica di una legge di distribuzione. G. Ist. Ital. Attuari. 1933; 4:83-91
7. Smirnov N. Table for estimating the goodness of fit of empirical distributions. Annals of Mathematical Statistics. 1948; 19:279-281
8. Jang S, Lee I, Weimer J. Improving classifier confidence using lossy label-invariant transformations. Proceedings of The 24th International Conference on Artificial Intelligence and Statistics, PMLR 2021; 130:4051-4059

Although specific examples and features have been described above, these examples and features are not intended to limit the scope of the present disclosure, even where only a single example is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed in this specification (either explicitly or implicitly), or any generalization of features disclosed, whether or not such features or generalizations mitigate any or all of the problems described in this specification. Accordingly, new claims may be formulated during prosecution of this application (or an application claiming priority to this application) to any such combination of features. In particular, with reference to the appended claims, features from dependent claims may be combined with those of the independent claims and features from respective independent claims may be combined in any appropriate manner and not merely in the specific combinations enumerated in the appended claims.

What is claimed is:

1. A method for detecting stroke, the method comprising:
receiving, at a stroke detector implemented on at least one processor, movement data from an accelerometer attached to an upper limb of a patient for a period of time;
analyzing, at the stroke detector, the movement data using a test statistic robust to motion distribution covariate shift to enable passive monitoring of the patient; and
outputting, at the stroke detector, an alarm signal in response to detecting a stroke using the movement data.

2. The method of claim 1, wherein analyzing the movement data using the test statistic comprises analyzing the movement data using parameter invariant (PAIN) statistics.

3. The method of claim 2, wherein the test statistic is a Komogorov-Smirnov statistic.

4. The method of claim 1, wherein receiving the movement data comprises receiving the movement data by a first wireless signal from a first wrist-mounted accelerometer on a first wrist of the patient.

5. The method of claim 4, wherein receiving the movement data comprises receiving a second wireless signal from a second wrist-mounted accelerometer on a second wrist of the patient.

6. The method of claim 1, wherein receiving the movement data comprises pre-processing the movement data to remove the effect of rotation/sliding of the accelerometer and bias.

7. The method of claim 1, wherein outputting the alarm signal comprises displaying an alarm message on a display screen.

8. A system for detecting stroke, the system comprising:
at least one processor; and
a stroke detector implemented on the at least one processor and configured to perform operations comprising:
receiving movement data from an accelerometer attached to an upper limb of a patient for a period of time;
analyzing the movement data using a test statistic robust to motion distribution covariate shift to enable passive monitoring of the patient; and
outputting an alarm signal in response to detecting a stroke using the movement data.

9. The system of claim 8, wherein analyzing the movement data using the test statistic comprises analyzing the movement data using parameter invariant (PAIN) statistics.

10. The system of claim 9, wherein the test statistic is a Komogorov-Smirnov statistic.

11. The system of claim 8, wherein receiving the movement data comprises receiving the movement data by a first wireless signal from a first wrist-mounted accelerometer on a first wrist of the patient.

12. The system of claim 11, wherein receiving the movement data comprises receiving a second wireless signal from a second wrist-mounted accelerometer on a second wrist of the patient.

13. The system of claim 8, wherein receiving the movement data comprises pre-processing the movement data to remove the effect of rotation/sliding of the accelerometer and bias.

14. The system of claim 8, wherein outputting the alarm signal comprises displaying an alarm message on a display screen.

15. A non-transitory computer readable medium storing executable instructions that when executed by at least one processor of a computer control the computer to perform operations comprising:
receiving movement data from an accelerometer attached to an upper limb of a patient for a period of time;
analyzing the movement data using a test statistic robust to motion distribution covariate shift to enable passive monitoring of the patient; and
outputting an alarm signal in response to detecting a stroke using the movement data.

16. The non-transitory computer readable medium of claim 15, wherein analyzing the movement data using the test statistic comprises analyzing the movement data using parameter invariant (PAIN) statistics.

17. The non-transitory computer readable medium of claim 16, wherein the test statistic is a Komogorov-Smirnov statistic.

18. The non-transitory computer readable medium of claim 15, wherein receiving the movement data comprises receiving the movement data by a first wireless signal from a first wrist-mounted accelerometer on a first wrist of the patient.

19. The non-transitory computer readable medium of claim 18, wherein receiving the movement data comprises receiving a second wireless signal from a second wrist-mounted accelerometer on a second wrist of the patient.

20. The non-transitory computer readable medium of claim 15, wherein receiving the movement data comprises pre-processing the movement data to remove the effect of rotation/sliding of the accelerometer and bias.
